(19) United States
(12) Patent Application Publication
(10) Pub. No.: US 2022/0001078 A1
(43) Pub. Date: Jan. 6, 2022

(54) THE SYSTEM OF AN ELEMENT USED FOR THE CREATION OF HEART VALVE, THE METHOD OF MANUFACTURING OF MODIFIED BACTERIAL CELLULOSE (BC), THE SET AND THE ELEMENT USED IN CARDIO SURGERY

(71) Applicants: GDANSKI UNIWERSYTET MEDYCZNY, Gdansk (PL); Bowil Biotech sp. z o.o., Wladystawowo (PL); Centrum Techniki Okretowej S.A., Gdansk (PL); Politechnkia Gdanska, Gdansk (PL); Uniwersytet Gdanski, Gdansk (PL); Fundacja Roczwoju Kardiochirurglii im. Prof Zbigniewa Religi, Zabrze (PL)

(72) Inventors: Piotr SIONDALSKI, Pregowo (PL); Magdalena KOLACZKOWSKA, Gdynia (PL); Waldemar WILANDT, Jurata (PL); Dariusz BOBINSKI, Jurata (PL); Aldona DLUGA, Jurata (PL); Kinga DAWIDOWSA, Bytów (PL); Michal DITRICH, Gdansk (PL); Leszek WILCZYNSKI, Gdansk (PL); Hanna STAROSZCZYK, Kraków (PL); Paulina DEDERKO, Bonin (PL); Edyta MALINOWSKA-PANCZYK, Gdynia (PL); Agata SOMMER, Reszel (PL); Izabela SINKIEWICZ, Pruszcz Gdanski (PL); Ilona KOLODZIEJSKA, Gdynia (PL); Marek SZKODO, Gdansk (PL); Alicja STANISLAWSKA, Gdansk (PL); Andrzej BORMAN, Gdansk (PL); Artur Hugo SWIERGIEL, Warzawa (PL); Paulina PALCZYNSKA, Gdansk (PL); Grzegorz JABLONSKI, Olsztyn (PL); Wojciech GLAC, Wejherowo (PL); Piotr WILCZEK, Jaworzno (PL); Maciej GAWLIKOWSKI, Zabrze (PL)

(21) Appl. No.: 17/291,248

(22) PCT Filed: Nov. 5, 2019
(86) PCT No.: PCT/PL2019/000100

§ 371 Date: May 4, 2021

(51) Int. Cl. A61L 27/36; A61L 27/20; A61L 27/50

(52) U.S. Cl. CPC A61L 27/3637; A61L 27/20; A61L 2430/20; A61L 27/507; A61L 27/3691

(57) ABSTRACT

The subject of the invention is the system of an element used for manufacturing heart valve, the method of manufacturing of modified bacterial cellulose (BC) with the use of Gluconacetobacter xylinus strain for the production of an element used for manufacturing heart valve, the set for implantation and the application of the element in cardio surgery.

The system of an element used for the creation of heart valve, the method of manufacturing of modified bacterial cellulose (BC), the set and the element used in cardio surgery.

The subject of the invention is the system of an element used for the creation of heart valve, the method of manufacturing of modified bacterial cellulose (BC) with the use of Gluconacetobacter xylinus strain for the production of an element used for the creation of heart valve, the set for implantation and the element use in cardio surgery.

Prostheses of heart valves may be manufactured from artificial materials—then they are known as mechanical prostheses, and from biological materials—then they are known as biological prostheses.

For patients with valvular defects, for who all methods of conservative treatment were applied, the only way of therapy is the implantation of heart valve prosthesis.

Problems related to the implantation of these protheses are above all else: degeneration of biological prosthesis and clotting on mechanical prosthesis. The valve built of BC is a solution of these problems, for what was proved by the researches conducted, on its surface no clotting takes place and this material is characterised by a high durability in comparison to natural tissues.

Prostheses currently available on the market are very expensive, so in the effect the choice is not only dependent on the biological qualities but also on costs related to implantation of various prostheses. The solution proposed by us gives the doctor and the patient a possibility of application of a cheap and safe treatment.

The currently known and used valves possess multiple faults. They require and additional treatment, i.e. anti-clotting treatment, they are subject to degeneration and therefore a new material is being searched for.

The descriptions of inventions present i.a. an elastic stent of biologically joined heart valves which allows a concurrent implantation of biological prosthesis of aortic valve and mitral valve—PL 180925 B. A European description of the invention EP 1083845 B presents an upgraded tricuspid mechanical valve, in which the casing of the valve has an articulated mechanism, what allows the spinning of cusps and their supporting. Another invention description PL 229562 B presents a mechanical tricuspid heat valve.

The utility of BC in healing of various wounds was already proven in the nineties. In relation to wound dressing and superficial/surface application BC is a non-toxic, non-irritating, hypoallergic, non-pyrogenic and biocompatible material.

BC may become the material used for manufacturing of durable, inner implants (vascular prostheses and heart valve prostheses), used especially in vascular surgery and cardio surgery.

Bacterial cellulose is a non-toxic, non-irritating, hypoallergic, non-pyrogenic and biocompatible material. It is characterised by a high mechanical strength.

There are many attempts of substituting of the currently used cardio-vascular implants, made from zoonotic and artificial materials, by BC membrane prostheses.

The great value of BC is its impermeability even for the slightest blood cellular elements (the diameter of thrombocytes reaches to the values of 1000-2000 nm and the size of the bio cellulose fibre net reaches from 20 to 100 nm). The surface of BC is therefore completely smooth for blood what decreases the possibility of a creation of clotting. This attribute should also affect the impossibility of outgrow of BC through the own cells of the host organism.

To date, the inventions concerning the way of BC in situ modification in situ were described, and this modification method is based on the application of chemical additives to the culture medium. In biomedicine these inventions are applied most frequently as new generation dressing materials especially in hard to heal wounds and as a material substituting natural tissues, i.a. cartilage tissue.

The description of patent PL 190961 B1 presents a manner of obtaining of the modified BC membrane, by adding the culture medium of poly-amino-saccharides, especially chitosan, in a microcrystalline form, of an average molecular weight of 20500 kDa and deacetylation degree of 70-95%, in quantity of 0.1-30 parts by weight. BC membranes obtained according to the invention possess glucosamines in chain, created in the effect of degradation of poly-amino-saccharides or their derived substances. The composite material derived in this manner is characterised by bioactivity and biocompatibility desired for biomedical application and it is biodegradable.

The description of patent application EP 346507 A reveals the manner of manufacturing a modified BC, through application carbomethylcellulose (CMC) as additive to the culture medium, in a form of used in the quantity of 0.1%-5% w/v. BC membranes created in the presence of CMC are characterised by higher dry and wet weight, as well as the increased water absorbency in comparison to BC membranes synthetized without the addition of CMC.

In patent PL 214844 B1 the method of modification of BC membranes based on enriching of culture medium by 0.25-1% CMC, and then, after the end of cultivation, by oxidization of membrane with 1% solution of sodium periodate in the temperature of 22° C. for 2 hours is presented. The membranes obtained are characterised by the increased water absorbency and a higher water retention capacity, in the effect of what a thicker, better hydrated material is created.

From the patent application US 2009/0220560 description we know a dressing obtained from BC, coated with nano silver, which gives the polymer antibacterial properties. The invention reveals obtaining of the cellulose fibres with the use of Acetobacter xylinum BPR 2001 strain. After cleaning, the cellulose fibres are treated, in this order, with 0.16 M sodium periodate, 1% amino thiourea in acetic acid, 1% silver protein in 2% sodium borate and silver salt in ammonia water at temperature of 95° C. The nano silver molecules formed as a result of a series of reactions cover BC fibres.

In the description of patent PL 216702 B1 the method of manufacturing of biomaterial with cartilage properties with the use of BC obtained in the process of cultivation of G. xylinus bacteria on a stationary medium. After obtaining and cleaning the BC membrane is modelled into a spatial structure of the desired shape and subjected to modification based of treating with 30% aqueous sodium lye solution, rinsing in distilled water, 10% aqueous acetic acid solution and repeated rinsing in distilled water until the cellulose material reaches pH 5.6-6.8. The material obtained this way has a very high mechanic strength, corresponding to the natural cartilage tissue, is biocompatible with human body and is non-allergic.

For applications in cardio surgery and vascular surgery, the materials manufactured as described above are unsuitable because they may be characterised by reduced human biocompatibility, inadequate mechanical properties, inadequate morphology and accelerated biodegradability.

In the description of patent application WO2016083352 (PCT/EP2015/077464) a heart valve prosthesis built up on a frame stent with leaflets made of particularly treated BC is presented. The method of preparing cellulosic material described in patent application WO2016083352 consists in producing a molded body made of bacterial cellulose, including the steps of mechanically pressing parts of the parts of molded body at temperatures in the range of 10° C. to 100° C. and pressing in the range of 0.0005 to 1.5 MPa by 10-200 min, treatment of the produced material with a solution consisting of: 20 weight % to 50 weight % glycerol and 50 weight % to 80 weight % alcohol/water mixture and drying the treated molded body. This technique is different to the method described in the present application for modifying the cellulose film which consists in initial drying and additional final soaking of the prepared structure in endotoxin-free water. The additional soaking process of previously dried cellulosic material is important and has a significant impact on its strength parameters.

In the description of patent application WO2013119912 (PCT/US2013/025287) a heart valve prosthesis molded on an expanding frame is presented. The cellulose material used as a valve described in the patent application number WO2013119912 is a composite system—cellulose material coupled to the frame of a defined structure. In addition, composite materials based on bacterial cellulose with the addition of silicone in a ratio of 60:40 (% by weight) have been described. Claim 22 describes a method of forming cellulosic material in a glass container by pouring a cellulose-based mixture.

In the description of patent application US 2009/0222085 A1 a heart valve molded on a stent as well as a particular BC treatment are presented. The description in the US Patent application number 2009/0222085 A1 relates to a method of modifying cellulosic material of plant origin in order to obtain a structure suitable for producing a heart valve. While it is important whether the cellulose is of plant or bacterial origin because of the chemical and mechanical properties that the particular material possess.

The method of modification of BC membrane in order to use it as a material for the production of bio prostheses in the circulatory system, obtained during stationary cultivation with the use of G. xylinus strain is characterised, according to the invention, that sterile BC membranes are dried to a constant mass at room temperature of not more than 25° C., then soaked in sterile distilled water at room temperature for no more than 120 minutes and stored under sterile conditions.

A variation of the invention is the BC membrane dried to a constant mass at room temperature of no more than 25° C. and then exposed to UV-C radiation with a total power of 12W and a maximum emission of 254 nm for a period of no more than 30 minutes. After exposure, the membranes are soaked in sterile distil water at room temperature for a period not exceeding 120 minutes and stored at sterile conditions.

Due to the use of the modification method according to the invention, it is possible to obtain biocompatible material with properties comparable to natural tissues building walls of blood vessels or valves, used in cardio surgery and vascular surgery for the production of bioimplants. In contrast to unmodified membranes, the material modified according to the invention is characterized by a water content similar to natural tissues and much higher mechanical strength. The material obtained according to the invention also has increased resistance to degradation processes in the human body.

The heart valve forming element is made of one sheet of biocompatible material, favourably polymer, favourably BC and is a flat, favourably biconnected figure, favourably with three axes of symmetry intersecting at one point, with a centrally made hole with three equal sides, not necessarily straight, forming a figure with symmetry analogous to that of an equilateral triangle. The axes of the symmetry of the element and the hole coincide. The edges of the hole are the first internal sides of the first areas of the element, and the first areas of the element being essentially quadrilateral in shape. Between the first areas of the element there are, directly adjacent to them, second areas of the element, whose outer edges are favourably rounded convexly. The second sides of the first areas are generally perpendicular to the first sides of the first areas, with the ratio of the length of the first side of the first area to the second side of the first area being between π/3 and 2π/3. The measure of the angles between the second and third sides of the first areas is 2π/3 to 5π/6. The length of the outer sides of the first areas is at least equal to the length of the first, inner sides of the first areas. The ratio of the radius of the circle coinciding with the centre of the symmetry of the hole and tangent to the outer side of the first area at mid-height to the length of the second side of the first area shall be between 2769 and 5π/6.

The aim of the invention is to provide a new heart valve and a new method of BC modification as the material of which the valve is made of. Moreover, the invention also aims to use the new valve in cardiac surgery.

The subject of the invention is the system of an element of the heart valve component which contains:
a flat sheet of BC with three axes of symmetry, with a centrally made hole (1) with three equal sides, forming a figure with the symmetry of an equilateral triangle,
the edges of the hole (1) are the inner sides of the first areas of the element,
the first areas of the element are quadrilateral,
between the first areas of the element are the adjacent second areas of the element,
the second sides of the first areas are perpendicular to the first sides of the first areas,
the angle between the second and third sides of the first areas shall be 2π/3 to 5π/6,
the length of the outer sides of the first areas shall be at least equal to the length of the first inner sides of the first areas,
the ratio of the radius of the circle coinciding with the centre of symmetry of the hole and tangent to the outer side of the first area at mid-height to the length of the second side of the first area shall be between 2π/9 and 5π/6.

The element is positively a biconnected figure.

The element where the axes of symmetry intersect in one point.

The element where the axes of symmetry of the element and the hole overlap.

The element where the outer edges of the second areas are rounded convexly.

The element where the ratio of the length of the first side of the first area to the length of the second side of the first area is between π/3 and 2π/3.

The element where the first areas (I) meets the second areas (II) on the outer side of the element has strengthening knobs (2) favourably semi-circular.

The subject of the invention is the method of manufacturing of the modified BC with the use of the G. xylinus strain for manufacturing of the element defined above, where:
sterilized BC is dried to constant weight at room temperature not exceeding 25° C.,
is sterilized at 121° C. for 20 minutes,
the obtained BC is stored under sterile conditions.

The method in which dried BC is exposed to UV-C radiation using lamps with a total power of 12 W and maximum emission at 254 nm, for a period of 30 minutes, while maintaining sterility.

The method in which dried BC is soaked in sterile distilled water at room temperature for up to 120 minutes and stored in refrigerated conditions while remaining sterile.

The method where, after exposure, it is soaked in sterile distilled water at room temperature for up to 120 minutes and stored in refrigerated conditions with sterility.

The subject of the invention is also an implantation kit, which contains the element specified above for use in cardio surgery.

The element defined in claim 1 for the use in cardio surgery.

### DESCRIPTION OF THE FIGURES

FIG. 1—shows an element for creating the valve

FIG. 2—shows the element for creating the valve

FIG. 3—shows an element for creating a valve with cusps

FIG. 4A—shows a diagram of areas of the element marked on FIG. 1 and/or FIG. 2 with number II, which are “T” shaped overlaps, by connecting them with the wall of the tube.

FIG. 4B—shows a diagram of areas of the element marked on FIG. 3 with number II, which are “T” shaped overlaps, by connecting them with the wall of the tube.

FIG. 5a—represents the tensile strength held in physiological saline solution

FIG. 5b—represents the tensile strength fixed in glutaraldehyde

FIG. 5c—shows the tensile strength of: BC native sheets; composites: BC-polyvinyl alcohol PVA, BC-hyaluronic acid;

FIG. 5d—represents the tensile strength: BC panels modified: BC dry s, BC 9 days, BC 8 days

FIG. 5e—represents the tensile strength: BC panels modified: BC with added aminobac.

FIG. 6—shows the values of elasticity modulus and strain in the breaking test measured in the peripheral and axial direction.

FIG. 7—shows the values of breaking energy measured in the axial and peripheral direction.

FIG. 8—presents hysteresis test before dynamic fatigue test

FIG. 9—shows the hysteresis test after the dynamic fatigue test.

FIG. 10—shows the X-ray of the valve implanted to sheep no. 1261

FIG. 11—shows the X-ray valve implanted to sheep no. 4584

FIG. 12—shows the valve in histopathological examination.

FIG. 13—shows the determination of calcium content in samples.

The invention is illustrated by the following examples, which do not constitute its limitation.

### Example 1

Sterilized BC membranes were laid on a flat surface and dried to constant weight at room temperature not exceeding 25° C. (convection drying, carried out in a dryer without forced circulation at 25° C. The drying agent was atmospheric air). The membranes were then packed and sterilized in autoclave at 121° C. for 20 minutes. Dried BC membranes were stored sterile.

### Example 2

1 BC membranes dried as in the example 1 were exposed to UV-C radiation with lamps of a total power of 12 W and maximum emission at 254 nm. Dried BC membranes were placed 5 cm away from the lamp and exposed for 30 min. BC membranes were stored sterile.

### Example 3

BC membranes prepared as in example 1 were soaked in sterile distilled water at room temperature for up to 120 minutes and stored in refrigerated conditions, sterile.

### Example 4

BC membranes prepared as in example 2 were soaked in sterile distilled water at room temperature for up to 120 minutes and stored in refrigerated conditions, sterile.

### Example 5

The heart valve component is made of a single sheet of biocompatible material, for example BC, as shown in FIG. 1. The edges of the central hole 1, which is an equilateral triangle, are the first sides A of the first areas of the I component. The first areas of the I element are similar in shape to a quadrilateral, whose outer edge is convexly rounded. The arc of this rounding at its highest point is tangential to a circle having a radius R equal to the length of the edge A, and of the centre coinciding with the centre of the central hole 1. Between the first areas of the I component there are immediately adjacent second areas of the II component whose outer edges are convexly rounded. The second H sides of the first areas I are perpendicular to the edge A of the central hole 1. The measure of the angle β between the second sides 2 is 2π/3 and the relationship between the length of the edge A and the length of the second H side of the first area I is

$$H = \frac{2 A}{π} .$$

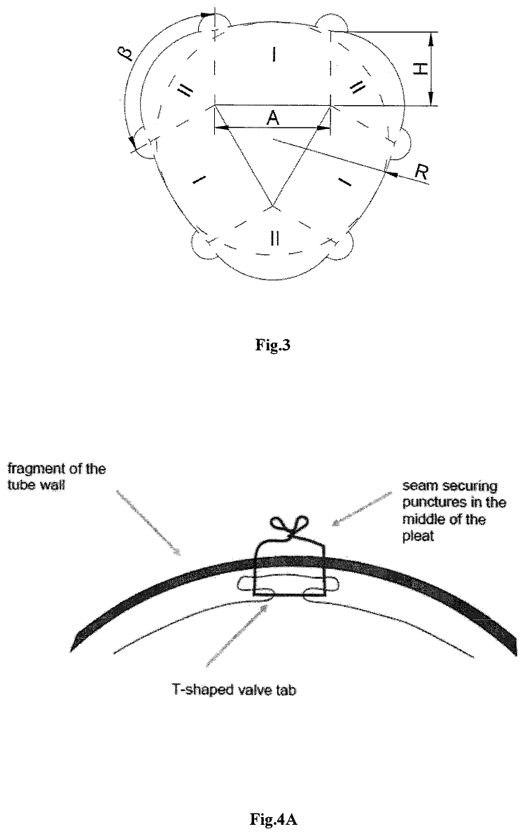

From the element created by the suitable, known anastomosis, for example a stitching, the valve is formed in a way that the second areas of the second element bends outwards and forms T-shaped fasteners in the tube in which the valve is to act, as shown in FIG. 3 in top view

$$R = A \\ β = \frac{2}{3} π \\ \frac{A}{H} = \frac{1}{2} π \\ \frac{R}{H} = \frac{A}{H}$$

| Parameter | | Example 5 |
|---|---|---|
| A | [mm] | 21.0 |
| R | [mm] | 21.0 |
| H | [mm] | 13.4 |
| β | [rad] | 2.09 |

### Example 6

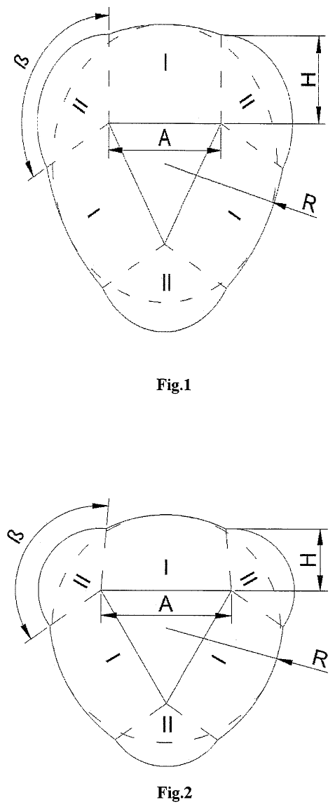

The element for creating the heart valve as shown in FIG. 2 is manufactured as in example I, but for an angle of β of 2π/3, the relationship between the radius of the circle R and the length of the edge A of the central hole 1 and the length of the other side H are:

$$H = \frac{3 A}{2 π}$$

$$R = \frac{7 A}{8}$$

$$β = \frac{2}{3} π$$

From the element created by the suitable, known anastomosis, for example a stitching, the valve is formed in the way that the second areas element II are bended outwards and forms T-shaped fasteners in the tube in which the valve is to act, as shown in FIG. 3 in top view

| Parameter | | Example 6 |
|---|---|---|
| A | [mm] | 29.3 |
| R | [mm] | 34.3 |
| H | [mm] | 18.7 |
| β | [rad] | 2.09 |

### Example 7

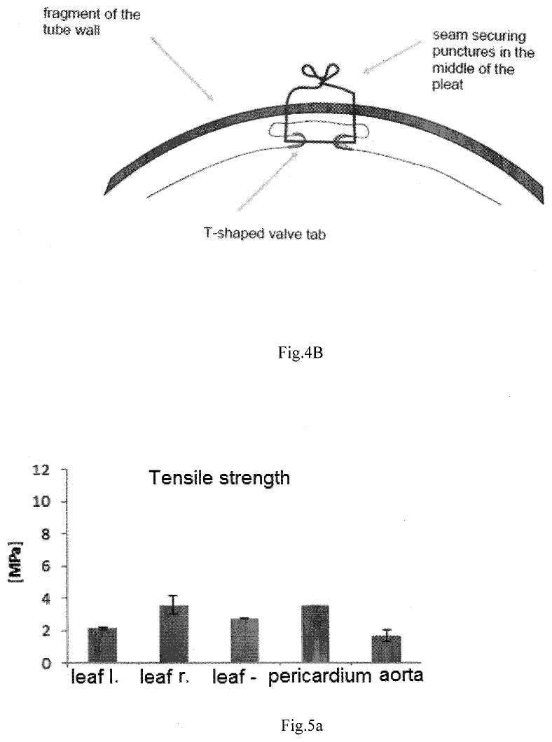

The element as in the example 5 was cut out from BC obtained as in the example 1-4, additionally forming the C amplifying cusps. During the stitching process, the reinforcing cusps C are bent downwards, to the outer side of the valve, as schematically shown in FIG. 4B.

### Example 8

The suture method involves determining three points every 120 degrees, on the circumference of the circle in the tube in which the valve is to act. This ring will be the place where the edges of the central opening of the element will be connected with the tube in which the valve will function. The tube may be made of artificial material or natural tissues.

The connection between the edges of the central opening and the ring may be made by continuous or interrupted surgical suture.

The areas of the element marked on FIG. 2 number II will constitute “T” shaped overlaps by connecting them with the wall of the tube according to the diagram in FIG. 3. The height of this connection is determined by H, counted from the points of stitching of the corners of the central hole upwards on the wall of the tube, evenly located above the points of the corners.

In this way the lower floor of the connection between the valve inside the tube's light and the element remains completely tight—impermeable to liquid, and the stitching of “T” shaped tabs from the top will cause the liquid flowing from the top to the bottom in the tube will cause a tight adherence of the areas of the element numbered I to FIG. 2. The liquid flowing inversely will cause the free opening of the areas I and thus the free flow of the liquid only in this direction.

### Example 9

Modifications of BC aiming at obtaining a material resistant to enzymes and selected pathogenic microorganisms and determining of the influence of these modifications on other properties of the polymer.

Resistance of modified BC to in vitro degradation was investigated by determining the changes occurring in the polymer during incubation of samples at 37° C. in a solution simulating physiological fluids in the absence and presence of Aspergillus fumigatus.

Modification of BC:

Sterile BC membranes, in the shape of squares of 2.5×2.5 cm and strips of 1.5×10 cm, modified by drying at room temperature and soaking in water and by drying at room temperature.

The dried cellulose membranes were additionally modified by UV radiation using two low-pressure mercury lamps, each of a power of 6 W and a maximum emission at 254 nm. The samples were placed at a distance of 5 cm from the lamps and exposed for 30 minutes, after which they were soaked in sterile distilled water for 60 minutes. The modification was carried out with sterility.

BC samples (measuring 2.5×2.5 cm) modified by drying at room temperature and soaking in water and by drying at room temperature, exposed to UV radiation and soaked in water were placed in sterile bottles of 100 mL filled with 62.5 mL of sterile SBF liquid. For the study of mechanical properties, modified BC strips (1.5×10 cm) were placed in 150 mL of the solution in sterile bags. A sufficient amount of A. fumigatus liquid mould culture was added to a part of the samples immersed in SBF solution so that the initial number was about 10³ cfu per 1 mL of liquid. Degradation changes of BC samples were examined after 3, 7, 14, 30 days from the beginning of incubation.

Determination of BC Biodegradability Degree:
1. determination of the BC wet mass changes
2. examination of mechanical properties of BC
3. examination of thermal and structural properties of BC
4. X-ray diffraction analysis (XRD)
5. microscopic analysis using SEM technique

The results of the experiment were developed using the statistical program SigmaPlot 11.0 (SYSTAT Software, Germany), with the help of analysis of variance with ANOVA single-factor classification for significance level p<0.05.

Wet mass of BC samples modified by drying at room temperature and soaking in water and then stored in sterile SBF fluid for 3, 7 and 14 days increased twice, and after 30 days of incubation no statistically significant differences were observed. In case of samples incubated in the presence of A. fumigatus, greater changes in wet mass were observed than in case of BC samples stored under sterile conditions. After only 3 days of incubation the wet mass increased about 2.5-fold, and after 7 days 4-fold.

Different results were obtained during storage of BC samples modified by drying at room temperature, UV radiation and soaking in water. In this case, incubation in sterile SBF fluid resulted in a decrease in the content of wet BC mass by approx. 60% (FIG. 3), and incubation in SBF fluid in the presence of A. fumigatus resulted in a decrease in this content by approx. 50% only after 3 days of incubation.

The stress at a break (σ) of non-incubated BC samples modified by drying at room temperature and soaking in water was about 112 MPa and the relative elongation at break (ε) about 13%. In case of samples modified by drying at room temperature, UV radiation and soaking in water, σ of the non-incubated membranes was about 160 MPa and ε about 9%.

Storage both in sterile SBF fluid and in the presence of A. fumigatus deteriorated the mechanical strength of BC membranes modified by drying at room temperature and soaking in water. Already after 3 days of incubation in sterile SBF fluid a decrease in a by about 60% was observed, after 7 days by about 20%, and after 14 and 30 days by about 50%. Incubation in the presence of A. fumigatus for the period of 3 days caused a similar decrease in a as in the case of mould-free incubation by about 60%, but after 14 and 30 days a decreased significantly more, by 70 and 90% respectively. c of the samples did not change during the whole incubation period in sterile SBF fluid, whereas in the presence of A. fumigatus it decreased by about 30% after 14 days of incubation and by about 70% after 30 days. The values of σ and ε of BC samples dried at room temperature, exposed to UV radiation and soaked in water and then incubated in sterile SBF fluid did not change in comparison to non-incubated samples, except for c samples stored for 14 days, value of which increased by about 20%. On the other hand, BC samples incubated in SBF fluid in the presence of A. fumigatus showed about 20 and 60% lower a value after 7 and 30 days of incubation respectively, in comparison to radiated and non-incubated samples. c of these samples did not change, except for samples incubated for 14 days, which increased by about 30%.

When analysing the diffractograms of all modified BC samples, 3 characteristic diffraction bands were observed at the reflective angle of 2θ 14.46°, 16.7° and 22.62°. The incubation of the samples under the conditions simulating body fluids did not affect the position of diffraction lines but caused a change in the intensity of diffraction bands at the angles of reflection 2θ 14.46° and 22.62°. Additionally, it was noted that in the case of BC diffractogram dried at room temperature and soaked in water and then stored for 3 days in both sterile SBF and liquid SBF in the presence of A. fumigatus, the intensity of the diffraction line is increased at an angle of 2θ 16.7°. The crystallinity degree (Cr.I.) of all modified BC samples calculated on the basis of the formula of Segal et al. (1959) was about 90%.

Changes in BC crystallinity caused by biodegradation of samples modified by drying at room temperature and soaking in water, drying at room temperature, UV radiation and soaking in water were compared. The crystallinity degree of all tested samples differed only slightly. The exception was BC dried at room temperature and soaked in water and then kept for 3 days in the presence of mould. In this case, a decrease in Cr.I. by about 5% was observed in comparison with the non-incubated sample.

The decomposition temperature of BC that is not incubated, dried at room temperature, UV-radiated and soaked in water was approximately 6° C. lower than that of BC that is dried at room temperature and soaked in water. A lower decomposition temperature indicates lower thermal stability of the material, probably due to greater susceptibility to biodegradation of the material. SEM images of the surface of all BC samples modified and then incubated in sterile SBF fluid did not show any differences compared to the surface of non-incubated samples. Hardly visible, thin fibres can be observed on the sample surface. A more compact structure of BC samples incubated in sterile SBF fluid may be the reason for its increased mechanical strength in comparison with the samples incubated in SBF in the presence of A. fumigatus.

Obtaining BC with greater resistance to biodegradation characteristic for bio-prostheses of blood vessels and aortic valves.

### Example 10

Mechanical testing of BC and BC-based composite materials.

The conducted research allowed to choose BC with no worse tensile strength than natural tissues of the swine cardiovascular system. In order to achieve this, BC sheets of 10×1.5 cm in size were subjected to a tensile test. The reference material were natural tissues: aorta, aortic valve and pericardial sac fragments. Natural tissues were properly prepared and supplied by the Medical University of Gdansk. The tissues were divided into two groups. The first group consisted of tissues stored in physiological saline solution, from the moment the samples were delivered to the moment the tensile test was performed. However, the second group—tissues additionally washed with 0.5% glutar aldehyde for 10 minutes prior to the tensile test.

The material is: Native BC, Modified BC and Composite BC-Polyvinyl alcohol, BC-hyaluronic acid.

The tensile test was carried out on an INSTRON model 1112 testing machine with a single-axis tensile velocity of 5 mm/s. The distance between the jaws for BC stretching was 50 mm.

Studies show that thermally modified BC, i.e. dried “s” and then soaked, has the best tensile strength of all bio-nanocellulose materials and has a strength of 22 MPa. Native (homogeneous) BC and BC-based composite materials have a tensile strength of about 5 MPa, which is lower than that of natural materials. The value of approx. 22 MPa corresponding to thermal modified BC allows for further strength testing (tear test, fatigue test) on selected BC material, which may be a potential material used in cardio surgery and vascular surgery.

The susceptibility of BC to in vitro degradation in simulated body fluid (SBF—at 37° C.) was determined. Various methods of monitoring degradation changes were applied, e.g. determination of dry and wet mass changes, determination of biomaterial hydrolysis products using liquid chromatography and thermal stability of BC. The surface of biomaterial was evaluated by scanning electron microscopy (SEM) and the development of microorganisms deliberately introduced into the environment in which BC was incubated was also monitored.

During the storage of BC samples in sterile SBF fluid and PSB buffer no changes in dry matter were found for the whole 6-month storage period. Furthermore, in the presence of S. aureus bacteria and C. albicans yeast, the dry matter of BC samples remained at a similar level. Significant decrease in BC dry matter was observed only in the samples incubated for 6 months in the presence of A. fumigatus mould—BC dry matter decreased by 41%. In case of wet mass, it was found that it significantly increased after the second month of storage, regardless of the conditions (PBS buffer, SBF liquid in the presence and absence of microorganisms).

Moreover, in the presence of S. aureus bacteria and C. albicans yeast, the dry matter of BC samples remained at a similar level. Significant decrease in BC dry matter was observed only in the samples incubated for 6 months in the presence of A. fumigatus mould—BC dry matter decreased by 41%. In case of wet mass, it was found that it significantly increased after the second month of storage, regardless of the conditions (PBS buffer, SBF liquid in the presence and absence of microorganisms).

It was also shown that during the storage of BC samples the growth of all tested microorganisms occurred. After 1 month, the number of S. aureus, C. albicans and A. fumigatus cells increased from about 10³ to 10⁵ cfu/cm³ and remained at the same level up to 6 months, which indicates that they were in the stationary phase of growth. The growth of microorganisms and wet BC mass indicates that degradation changes occur in this material, although its dry mass does not change significantly (except for the samples containing A. fumigatus moulds). It was also found that during the storage of samples on the BC surface only A. fumigatus moulds formed a biofilm on the surface. The concentration of saccharides, which are the products of BC hydrolysis, in the post-incubation fluids was so low that they could not be detected by thin layer chromatography (TLC). After a 20-fold concentration of these fluids, both from samples with and without S. aureus bacteria and C. albicans yeasts, no BC hydrolysis products were found. However, they were present (though in small amounts) in the concentrated post-incubation fluid after only one month of BC treatment with A. fumigatus moulds. Analysing the obtained results, it can be stated, however, that the investigation of the presence of cellulose hydrolysis products in post-incubation fluids is not a good method to determine the degree of polymer biodegradation, because microorganisms can metabolize simple sugars, disaccharides and oligosaccharides produced in this process.

The storage of samples in PBS buffer and sterile SBF fluid for the period of 5 and 6 months resulted in decrease in BC decomposition temperature by about 10° C., which indicates degradation processes and reduction of water adsorbed on its surface by half on average. Similar changes were observed in the samples stored in the presence of S. aureus bacteria and C. albicans yeasts. However, the samples stored in the presence of A. fumigatus mould for 6 months had thermal stability decreased by approx. 20° C. The analysis of thermograms of samples incubated in SBF fluid for 6 months in the presence and absence of microorganisms also showed an additional effect indicating the loss of chemically combined water.

Microscopic observations showed that the morphology of BC membrane surface after incubation in simulation fluids for the period of 1-6 months did not change significantly, it only became more porous.

### Example 11

The description of the tests that have been performed to assess the properties of BC. The aim of the task was to assess the biomechanical properties of BC material in vitro.

1. Tear Test

For each test, single-axis tear tests were performed using Tytron 250 Microforce Testing System (MTS) with a 250 N force transducer. Percentage strain was measured using a video extensometer (Messphysik). For proper strain analysis, appropriate markers were used to determine the L0 and L1 parameters of the specimen during breaking test. Before each test, the extensometer was calibrated at the use of standards provided by the manufacturer.

For proper measurement, it was attempted to maintain the width to length ratio of the specimen 1:10. Each time prior to the test, the thickness of the specimen was measured, which was an input to automatically obtain the cross-sectional area value and thus automatically recalculate the elasticity. In case of tensile tests, the specimen was preloaded with 0.5 N and the breaking test started from this value each time a test started.

The elasticity, percentage of real strain and breaking energy were measured. Taking into account the anisotropic character of BC material, the tests were performed in two conventional peripheral and axial directions. Prior to the tests, the tested material was weighed each time; it resulted from the fact that BC has strong hydrophilic properties, which allowed to obtain information on the extent to which the biomechanical parameters could be influenced by the degree of hydration of the sample.

2. Viscoelastic Properties Test

Viscoelastic properties of BC samples were also tested by hysteresis. Prior to the test, the specimens were preloaded to 1 N, then stretched to 4% strain and maintained for 60 seconds and then returned to their initial values. The hysteresis value was measured as the difference between the input and output energy.

3. Dynamic Fatigue Test

Dynamic fatigue test was performed at 370° C. in an environmental chamber filled with DMEM/F12 medium supplemented with 10% serum and antibiotics. Similarly to the tensile tests, BC samples were weighed before and after the completion of the test. The test was performed at 5 mm amplitude at 3 Hz at 250,000 cycles. At the end of the fatigue test, the hysteresis of the specimen was determined each time.

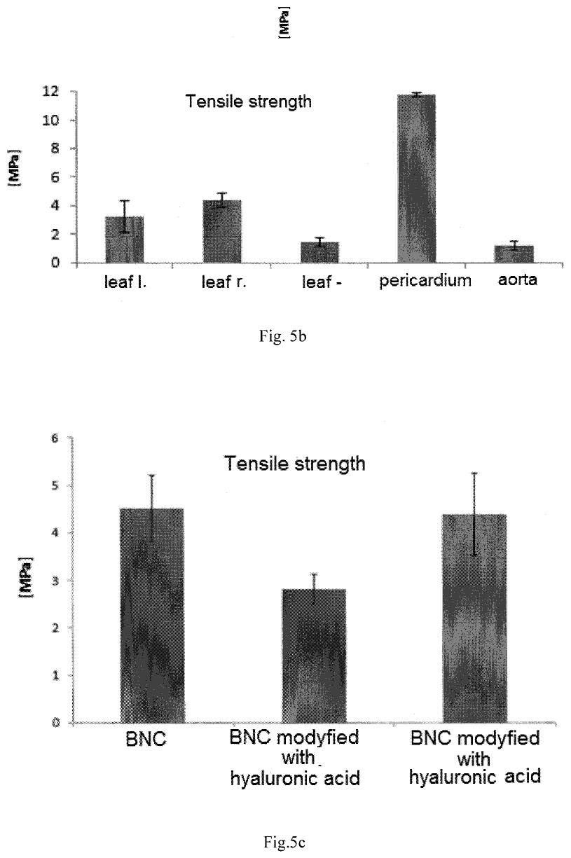

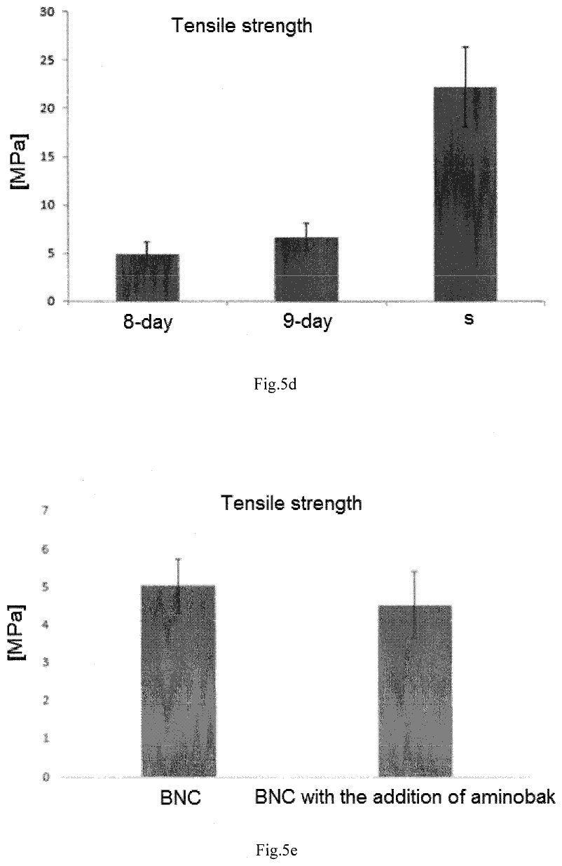

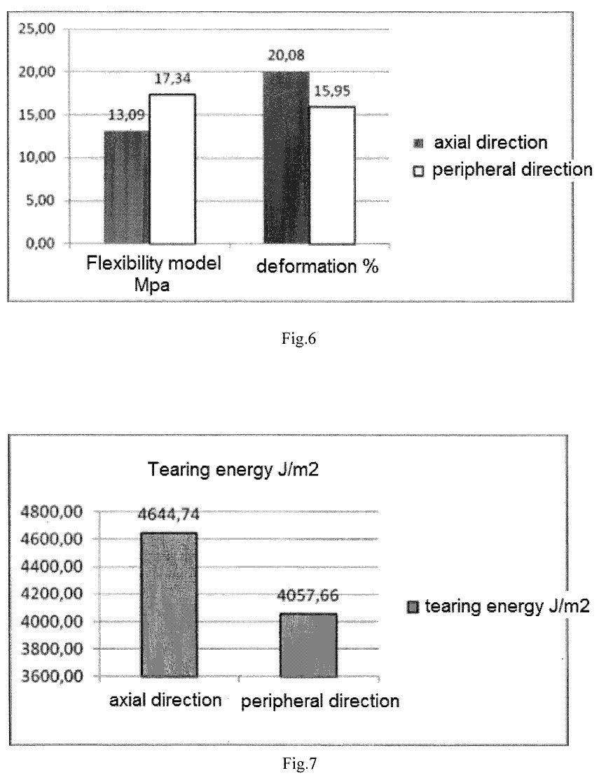

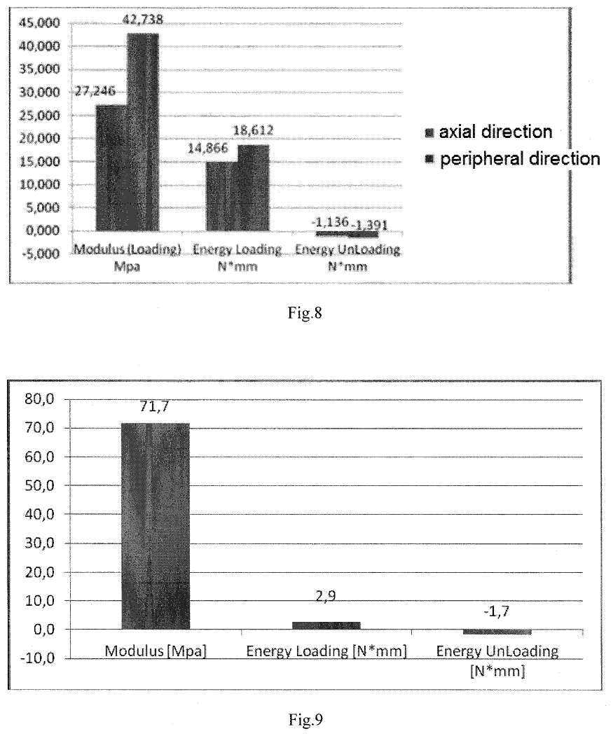

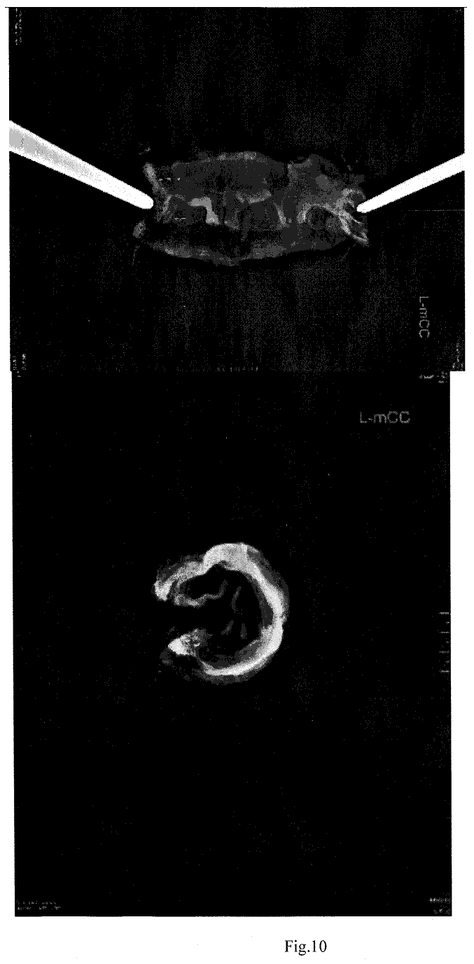

The elasticity, strain and breaking energy differed depending on the direction of the specimen arrangement (FIG. 5,6).

Hysteresis tests indicate that BC has poor viscoelastic properties. Due to the anisotropic character of the BC samples, the stiffness of the material was observed in a dynamic fatigue test (FIG. 7, 8).

Biomechanical properties indicate that the studied BC material has much higher stiffness in relation to human and swine tissues (pulmonary and aortic valves). The BC material has high hydrophilicity.

Features of the Obtained Valve:

For the material which is flat, flaccid and of the thickness between 300 and 600 micm as well impenetrable for the morphotic components of blood:

1. tensile strength exceeding 5 MPa

native 5 MPa, modified 22 MPa

Studies show that thermally modified BC, i.e. dried “s” and then soaked, has the best tensile strength of all bio-nanocellulose materials and has a strength of 22 MPa. Native (homogeneous) BC and BC-based composite materials have a tensile strength of about 5 MPa, which is lower than that of natural materials. The value of about 22 MPa corresponding to the thermal modified BC allows to continue the strength tests (tear test, fatigue tests) on the selected BC material, which may be a potential material used in cardio surgery and vascular surgery.

2. fatigue resistance with the following parameters:

force 45 N

vibration amplitude A=5 mm

frequency f=3 Hz

time 24 hours

3. biostability (after 6 months of incubation in sterile PBS buffer and sterile SBF fluid)

Storage of samples in sterile PBS buffer and sterile SBF fluid for the period of 5 and 6 months resulted in the decrease in BC decomposition temperature by about 10° C., which indicates degradation processes and reduction of water adsorbed on its surface by half on average. Similar changes were observed in the samples stored in the presence of S. aureus bacteria and C. albicans yeasts, whereas the samples stored in the presence of A. fumigatus mould for 6 months had a thermal stability decreased by approx. 20° C. The analysis of thermograms of samples incubated in SBF fluid for 6 months in the presence and absence of microorganisms also showed an additional effect indicating the loss of chemically combined water.

Microscopic observations showed that the morphology of BC membranes surface after incubation in simulation fluids for the period of 1-6 months does not change significantly, it only becomes more porous.

4. low hemolysis index

5. low trombogenicity

The study was performed on fresh heparinized swine blood obtained during traditional slaughter of animals in a slaughterhouse. A total of 13 experimental sessions were conducted—7 without BC (control) and 6 with the use of BC (experimental). In half-hour intervals, activated coagulation time (ACT), total haemoglobin (Hb), free haemoglobin (fHb), erythrocyte count (RBC) and haematocrit (HTC) were examined.

ACT monitoring was an element of the applied methodology—Schima test. This parameter, which is an indicator of the tendency of blood to coagulate, determines the moment of termination of the experiment and is not useful in itself for the assessment of the degree of hemolysis or thrombogenicity of the tested material. HCT, RBC and Hb changed slightly during the study and did not exceed the norms given in the literature for swines. fHb in plasma is closely correlated with the haemolysis process in the circulating medium which was studied. During the studies (both control and with the use of BC) it was subject to a gradual, systematic increase.

The comparison of fHb growth dynamics during control and experimental studies allowed to make a conclusion concerning the influence of BC on hemolysis. Since it is affected by both blood parameters (haematocrit, initial concentration of free haemoglobin) as well as pumping time, the obtained data required standardization. For this purpose, IH index (Free Haemoglobin Index) was used to determine the increase in plasma free haemoglobin content in mg/l of pumped blood. This is particularly important due to differences in the duration of individual trials. IH was calculated from the formula:

$$IH = \frac{Δ Hg * ( 100 - HTC )}{Q * T}$$

ΔHg [mg/l]—difference between the concentration of free haemoglobin in the plasma between the first and the last measurement

HTC [%]—Haematocrit

Q [l/min]—Flow

T [min]—Flow time

The mean (M±SD) haemoglobin index for control samples was equal to: IH=14.71±9.42, and for experimental IH=12.87±3.52, while the mean (M±SD) circulating medium flow time (M±SD) for control samples T=325.57±75.08 min., and for experimental T=330.00±60.75 min. At the same time the differences between these mean pairs (control/experiment) turned out to be statistically insignificant (p>0.05).

It is worth noting that despite a slightly longer time of pumping blood, a contact with BC resulted in a lower hemolysis index than for control samples.

Thrombogenicity testing according to the Schima protocol is limited to the assessment of the extent to which the surface of the tested material is covered by thrombi. Macroscopic evaluation of BC fragments after the completed experiments in most cases showed the presence of relatively small number of red thrombi, especially on the surface in direct contact with flowing blood. The number of observed thrombus was even lower on the “opposite” surfaces of scraps loosely adjacent to the walls of flow channels of the equipment.

The Schima BC test does not show a significant haemolytic activity and its thrombogenicity seems to be insignificant.

6. low adhesiveness

BC is characterized by low adhesiveness. Lack of proper adhesion of cells affects the generation of necrotic processes. Surface modification of BC with the use of natural proteins of intracellular matrix significantly improves the adhesiveness of cells and growth characteristic, what may have a significance in case of clinical application of BC.

### Example 12

Implantation of bio-nanocellulose valve performed in sheep Sheep numbers:

1261
4466
4584

All implants of the valves were manufactures according with the protocol. During the first week after the implantation of the valves (first post-operative week), no adverse events were reported. All three sheep kept their normal appearance, general health and were not feverish. The recovery occurred in so-called normal status.

Then the sheep underwent a planned indirect ECG (within the period of 3 months). The sheep maintained a very good clinical condition. All echoes show that the valves are in a good clinical condition. 6 months after the operation, the sheep underwent echocardiography again with further maintenance of good health condition. The echo was performed inside the thorax cavity and the explant. Good elasticity and the valve cusp movement were stated. Distinctively blocked or calcified valve cusp were not noted.

The results of the sheep's blood tests—no significant abnormalities were detected. No signs of blood cells damage, hemolysis or infection.

Summing Up:

Lack of significant insufficiency. Good movement of the cusps of the valve, good elasticity.

Micro CT Scan

Scans reveal new structural defects in the valve cusps. There is a presence of minimal calcification, mainly in sheep nr 4466. The lowest calcification is seen in sheep nr 1261. Due to hypertrophy of new tissues, there is a slight thickening of the wall and cusps both from the side of the tunica intima (inner) and also from the tunica externa (outermost).

The median calcium content is only 1.88 μg/mg tissue, between 0.48 and 9.63 (IQR 0.56-4.51). The above graph shows the mean+/−SE and 95% CI.

### SUMMARY

The valve functions properly in the pulmonary position, more than 6 months after the pulmonary valve replacement surgery.

No surgical failure was noted. No damage to the material occurred. Lack of evidence of blood or platelet damage.

Low gradients and minor insufficiency were observed.

Only slight, temporary external calcifications connected with the suture were observed.

Good general clinical condition of animals.

Histological result shows lack of damage to the material, lack of calcification in the materials, only minor external calcification towards the matrix.

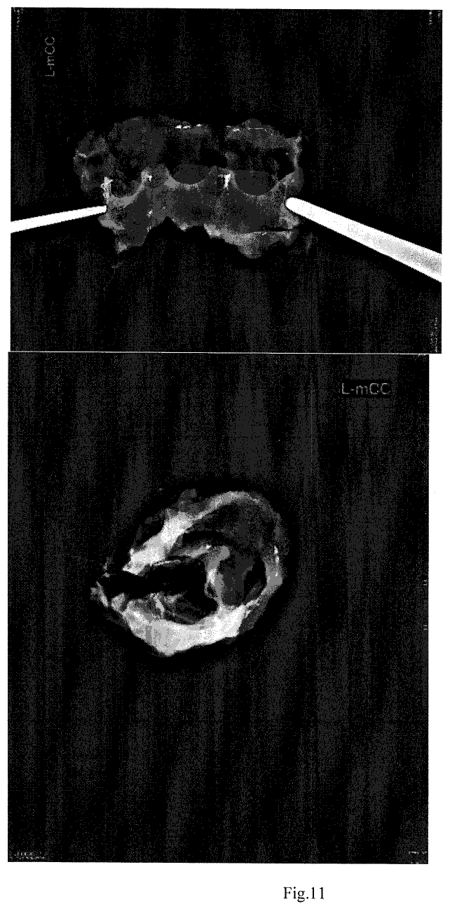

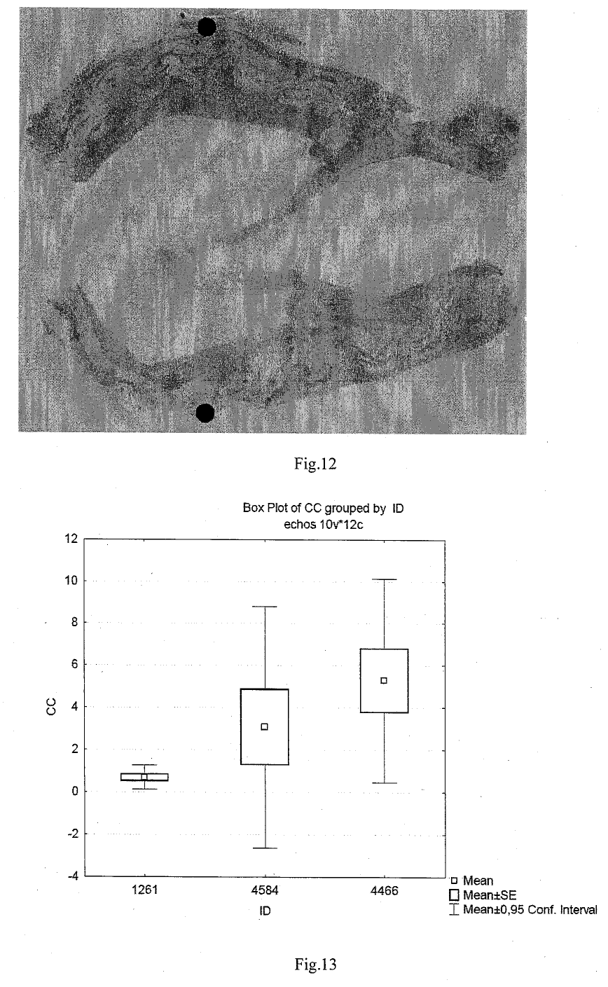

FIG. 10-13.

1. The system of an element of the heart valve component which contains:
a flat sheet of BC with three axes of symmetry, with a centrally made hole (1) with three equal sides, forming a figure with the symmetry of an equilateral triangle,
the edges of the hole (1) are the inner sides of the first areas of the element,
the first areas of the element are quadrilateral,
between the first areas of the element are the adjacent second areas of the element,
the second sides of the first areas are perpendicular to the first sides of the first areas,
the angle between the second and third sides of the first areas shall be 2π/3 to 5π/6,
the length of the outer sides of the first areas shall be at least equal to the length of the first inner sides of the first areas,
the ratio of the radius of the circle coinciding with the centre of symmetry of the hole and tangent to the outer side of the first area at mid-height to the length of the second side of the first area shall be between 2π/9 and 5π/6.

2. Element according to claim 1, is characterized in that it is a positively bioconnected figure.

3. Element according to claim 1, is characterized in that the axes of symmetry intersect in one point.

4. Element according to claim 1, is characterized in that the axes of symmetry of the element and the hole overlap.

5. Element according to claim 1, is characterized in that the outer edges of the second areas are rounded convexly.

6. Element according to claim 1, is characterized in that the ratio of the length of the first side of the first area to the second side of the first area is between π/3 and 2π/3.

7. Element according to any claims from 1 to 6 is characterized in that where the first areas (I) meets the second areas (II) on the outer side of the element has strengthening knobs (2) favourably semi-circular.

8. The method of manufacturing of the modified BC with the use of the G. xylinus strain for manufacturing of the element defined in claim 1 is characterized in that:
sterilized BC is dried to constant weight at room temperature not exceeding 25° C.,
is sterilized at 121° C. for 20 minutes,
the BC obtained is stored under sterile conditions.

9. The method according to claim 8 is characterized in that dried BC is exposed to UV-C radiation using lamps with a total power of 12 W and maximum emission at 254 nm, for a period of 30 minutes, while maintaining sterility.

10. The method according to claim 8 is characterized in that dried BC is soaked in sterile water distilled at room temperature for up to 120 minutes and stored in refrigerated conditions while remaining sterile.

11. The method according to claim 9 is characterized in that after exposure, it is soaked in sterile water distilled at room temperature for up to 120 minutes and stored in refrigerated conditions with sterility.

12. The set for implantation kit is characterized in that it contains an element defined in claim 1 for the use in cardio surgery.

13. The element defined in claim 1 for the use in cardio surgery.
